(19) United States
(12) Patent Application Publication
(10) Pub. No.: US 2011/0195515 A1
(43) Pub. Date: Aug. 11, 2011

(54) Capillary Plate and Method for Growth and Analysis of Macromolecular Crystals

(75) Inventors: Blattmann Beat, Richterswil (CH); Grutter Markus, Hochwald (CH)

(73) Assignee: UNIVERSITAT ZURICH, Zurich (CH)

(21) Appl. No.: 12/999,976

(22) PCT Filed: Jun. 3, 2009
(86) PCT No.: PCT/EP2009/056785

§ 371 Date: Dec. 17, 2010

(30) Foreign Application Priority Data
Jun. 20, 2008 (EP) 08011265.9

(51) Int. Cl. G01N 23/083; G01N 33/48

(52) U.S. Cl. 436/149; 422/68.1; 422/82.05

(57) ABSTRACT

A capillary plate and method for growing macromolecular crystals using the capillary plate. The capillary plate allows proteins and other macromolecules to be crystallized in the counter-diffusion method in a restricted geometry. Using this procedure, crystals can be adequately prepared for direct X-ray data analysis such that the macromolecule's three-dimensional structure can be solved without crystal manipulation.

### FIELD OF THE INVENTION

The present invention relates to a capillary plate and to a method for growing macromolecular crystals using said capillary plate. The capillary plate allows proteins and other macromolecules to be crystallized by the counter-diffusion method in a restricted geometry. Using this procedure, crystals can be adequately prepared for direct X-ray data analysis such that the macromolecule's three-dimensional structure can be solved without crystal manipulation.

### BACKGROUND OF THE INVENTION

Knowing the 3-D structure of biomolecules such as enzymes is vital to understand cellular processes.

The three techniques used for the structure determination of macromolecules are X-ray diffraction of protein crystals, nuclear magnetic resonance (NMR), and electron microscopy (EM). With X-ray crystallography being the most productive method to date.

Crystals of a protein are prerequisite for structure determination by X-ray crystallography. Crystallization of bio molecules is still a poorly understood process. The limiting step in protein structural determination is the ability to obtain protein crystals that are suitable for X-ray diffraction. Therefore, crystallization-screening methods have to be used to find a crystallization condition for a new protein. Using the common crystallization techniques, e.g., interface diffusion, vapour diffusion, batch under oil or lipidic cubic phase, a specific combination of condition variables has to be determined for each protein. Condition variables are e.g. the protein concentration, the temperature, time, pH, and the concentration of a wide range of precipitation agents in combination with various salts. High throughput screening in combination with miniaturizing the crystallization experiment to less than 500 nl is a practical solution for finding a suitable crystallization condition by using small amounts of purified protein.

The steps for obtaining crystals suitable for X-ray diffraction generally include determining the initial condition for protein crystallization, optimizing the initial condition to produce crystals suitable for X-ray diffraction, optimizing the treatment of crystal with cryoprotectant to allow the protein crystal to be frozen with liquid nitrogen, and as a final step screening for a strong X-ray scattering heavy metal ion that can incorporate into the protein crystal lattice without damaging the crystalline order to allow the phasing of the X-ray data.

Each step demands the use of extreme care when manually transferring the protein crystal between different solutions followed by mounting the crystal on a cryo-loop for X-ray analysis. Any inadvertent mishandling of the crystal damages the crystal structure, making the X-ray data collection impossible. The interface diffusion crystallization technique in glass capillaries eliminates the manual transfer of the protein crystal prior X-ray diffraction data collection and allows the measurement to be performed in-situ. Furthermore, this crystallization technique allows in-situ X-ray diffraction data collection of protein crystals too small to be manually handled with a cryo-loop. Current crystal recognition software is screening images automatically for protein crystals by searching within an image for shapes with straight contours. This crystal recognition approach so far has not been able to reduce false positive responses to a manageable level and, most important, to ensure no false negative results are created.

However, the methodological approach of the interface diffusion crystallization technique used in the prior art will not be able of reaching the high-throughput screening level needed for successfully making a wide spread use of this crystallization method. The capillary plate according to the present invention combines the high-throughput screening by interface diffusion crystallization technique in glass capillaries, with the in-situ X-ray diffraction data collection of protein crystals without any direct manual manipulation of the protein crystals. The images from a capillary plate taken with the help of an automatic incubation and imaging system, allow a novel approach of the functioning of a crystal recognition software by electronically subtracting a current image from the same image position taken at time zero whereby only an appearing crystal will produce a substantial signal and trigger an alert.

The technical problem underlying the present invention is to provide methods and devices for the crystallization of macromolecules in glass capillaries which combine the interface diffusion crystallization technique with high-throughput screening and with in-situ X-ray diffraction data collection of protein crystals.

The above technical problem is solved by providing the embodiments of the present invention as defined in the claims.

### SUMMARY OF THE INVENTION

In particular, according to a first aspect, the present invention provides a capillary plate comprising, within a support plate, at least one capillary tube
having a proximal and a distal end, and an inner diameter;
being adapted to receive a first liquid;
comprising at least one first region (“first sealing area”) and at least one second region (“second sealing area”) each being in contact with a means for fluid-tight closing the inner diameter of the tube within said first and said second region; and
comprising in between said at least one first and said at least one second sealing area at least one third region passing through a compartment for receiving a second liquid.

Preferably, the capillary plate of the invention has an at least one capillary tube comprising a fourth region between the at least one third and the first or the second sealing area, which fourth region, the so-called “inspection area”, is amenable to visible and/or optical inspection.

It is evident from the above that the above-defined regions of the capillary tube may together form a unit of sections of the tube having serving special purposes: the first and second regions provide means for fluid-tight closing the tube within this region, i.e. no fluid can pass this region of the tube, after the means for closing the tube in this region have been active, e.g. the inner diameter of the tube can be closed with a water immiscible sealing paste (which may be selected from silicone based vacuum grease, petroleum based vacuum grease, petroleum jelly, Vaseline®, Melkfett, paraffin, wax, clay, beeswax, dental wax, latex, polymer plugs, agarose and combinations thereof) present in a compartment, e.g. a well or a well-like structure, especially a well of dimensions typically encountered in microtiter plates, through which the capillary passes; or the tube may be heated in this area by a heating device such that the material of the tube melts within the first and second region, respectively. The third region, located between the first and second region, is a compartment through which the capillary passes and which can receive a liquid (the “second” liquid, since the capillary tube can, of course, receive also a (first) liquid, usually different from the second one). This compartment is also called herein-after as “precipitation reservoir”. Usually the above-described regions are arranged directly in sequence, preferably in the direction from the proximal to the distal end of the capillary tube. As outlined above, it is preferred that an inspection area is present as a fourth region of the capillary tube. This area is usually located between the compartment (“precipitation reservoir”) and the second sealing area. Within the inspection area it is possible to visibly monitor or to detect otherwise by optical means (e.g. microscope, optionally equipped with a CCD camera) changes in the state and/or contents of the liquid within the capillary tube.

The at least one capillary tube preferably comprises more than one of the above described units, more preferred several or a multitude of said units, e.g. from 16 to 96 unites, which are provided in a sequential manner. More preferably, said units are arranged in a rectangular array wherein groups of units, e.g. 3×16, 2×16, 6×16, 1×16, 6×16, 2×24 or 3×24 units, preferably form rows and columns (such that sealing areas, precipitation reservoirs and, optionally inspection areas are arranged in parallel; see FIGS. 2 to 11). With respect to the capillary tube this can be achieved in two ways: (i) the at least one capillary tube forms or follows a meander-like track within the support plate (see, e.g. FIG. 2a); (ii) there may be provided at least one capillary tube for every row of the rectangular array of said units such that the capillary tubes are arranged in parallel (see, e.g. FIG. 7b).

Preferably, each of said units is labeled (preferably individually) with an optical positional marker (hereinafter also denoted as “optical positional marker”).

The capillary tube has a proximal and distal end each of which may either be located inside or outside the support plate.

As already outlined above, the compartment(s) for receiving the second liquid (precipitation reservoir(s)) is/are preferably provided as well-like structure(s) through which the at least on capillary tube passes, preferably on or beneath its bottom area. More preferred is an arrangement in which the capillary tube is provided as a continuous polymer channel embedded or moulded in a polymer wafer. In this embodiment of the present invention it is preferred that at least an area of, preferably the complete bottom of each well (precipitation reservoir) is formed from a pierceable membrane.

According to another preferred embodiment of the present invention the at least one capillary tube is made of or comprises a flexible glass tube, more preferred made of quartz glass, borosilicate glass or silica. It is further preferred that the flexible glass tube is externally coated with a protective polymer which may be selected from the group consisting of standard polyimide, high temperature polyimide, acrylate, UV-transparent fluoropolymer, fluorinated acrylate, silicone, polyethylene, polypropylene, acrylic poly(methyl methacrylate), polystyrene, polyethylene terephthalate (PET), polycarbonate polymers, CR-39, copolymers of styrene, Nylon®, Teflon®, and their derivatives, and combinations thereof.

The capillary tube of the plate according to the present invention has preferably a round, more preferred a circular cross section, but it is evident that other geometries can be envisaged, e.g. triangular or rectangular or other cross sections, as long as the first liquid can enter the at least one capillary tube by capillary force. The latter constraint also guides the diameter of the at least one capillary tube.

The capillary plate according to the present invention is particularly useful for the examination of multiple probes of experiments making use of counter-diffusion, in particular for crystallisation purposes. Particularly for such applications it will be useful to equip the plate with means for high throughput and/or automatic processing. For example, the capillary plate may be mounted to a motorized x/y/z adaptor which can be in turn part of an X-ray diffractometer.

According to a second aspect, the present invention provides a method for crystallising biological molecules by counter-diffusion using the capillary plate according of the present invention comprising the steps of:
(a) filling the at least one capillary tube with a solution (first liquid) of the biological molecule;
(b) filling the individual compartment(s) (precipitation reservoir(s)) for receiving a second liquid with a solution intended to provide crystallization conditions for the biological molecule;
(c) interrupting and fluid-tightly closing the inner diameter of the at least one capillary tube within said first and second sealing areas; and
(d) forming a fluid connection between the compartments) (precipitation reservoir) containing the solution(s) (second liquid(s)) and the inner volume of the capillary tube (first liquid) in which crystallization of the biological molecule shall occur.

In the case of the preferred embodiment of the present invention in which more than one, preferably several or multiple units of first and second sealing areas, precipitation reservoirs and, optionally inspection areas, are present, it is preferred that each precipitation reservoir (compartment) receives a different solution (second liquid) such that multiple crystallisation conditions can be tested by using a single capillary plate of the present invention.

The method of the present invention is particularly carried out by using a capillary plate as defined above comprising multiple units of first and second sealing areas, precipitation reservoirs and, optionally, inspection areas, which units are preferably arranged in a rectangular array, thus comprising columns and rows of said units. Especially in this embodiment of the method according to the present invention, the above steps (c) and/or (d) are carried out for units belonging to the same row or to the same column of the rectangular array simultaneously.

As already outlined above, the capillary plate of the invention comprises at least one capillary tube preferably containing one or more inspection areas. In this case the method of the present invention comprises preferably the further step (e) in which the inspection area(s) is/are analysed, preferably visually and/or optically, for developing crystals of said biological molecule.

The step (e) is preferably performed with the aid of optical means, e.g. a microscope, preferably equipped with a CCD camera and corresponding image analysis software. In this context, it is especially preferred to take a first image from a location of said inspection area at a first time point and a second image from the same location of said inspection at a later, second time point. More preferred, the image information of said first image is then subtracted from the image information of said second image such that differences between the images can easily be detected.

### BRIEF DESCRIPTION OF THE DRAWINGS

The present invention is hereinafter described in more detail with reference to the accompanying drawings in which:

FIG. 1a shows a cross section of a flexible fused silica capillary tube referred to generally as 100. The flexible fused silica capillary tube 100 comprises the internal fused silica capillary tube 110, the external flexible transparent polymer coating 120, an inside diameter of the fused silica capillary tube 140, the outer diameter of the fused silica capillary tube 160, the wall thickness of the fused silica capillary tube 150, the thickness of the polymer coating 180 and the overall diameter 170 of the flexible fused silica capillary tube 100.

FIG. 1b illustrates the various dimensions of the polymer capillary tube 101, the polymer capillary tube 115, the inside diameter of the polymer capillary tube 145, the outer diameter of the polymer capillary tube 165, and the wall thickness of the polymer capillary tube 155.

FIG. 2a shows the top view of a capillary plate for 3×16 counter-diffusion experiments.

FIG. 2b shows the corresponding side view of the capillary plate shown in FIG. 2a.

FIGS. 2a and 2b illustrate an assembled capillary plate, the flexible fused silica capillary tube 100, the rectangular cuboid shaped support frame 200, the optical positional reverence points, referred to as optical registry markers or optical positional markers 205, the two ends of the flexible fused silica capillary tube 210 and 220, initial and final sealing areas 230, the plurality of precipitation reservoirs 240, and the horizontal crystal-observation window 250.

FIG. 3a illustrates the top view of a capillary plate for 2×16 counter-diffusion experiments.

FIG. 3b shows the corresponding side view of the capillary plate shown in FIG. 3a.

FIG. 3a and FIG. 3b illustrate an assembled capillary plate, the flexible fused silica capillary tube 100, the optical registry markers 205, the rectangular cuboid shaped support frame 200, the two ends of the flexible fused silica capillary tube 210 and 220, initial and final sealing areas 230, the plurality of precipitation reservoirs 240, and the horizontal crystal-observation window 250.

FIG. 4a illustrates the top view of a capillary plate for 1×16 counter-diffusion experiments.

FIG. 4b shows the corresponding side view of the capillary plate shown in FIG. 4a.

FIG. 4a and FIG. 4b illustrate an assembled capillary plate, the flexible fused silica capillary tube 100, the optical registry markers 205, the rectangular cuboid shaped support frame 200, the two ends of the flexible fused silica capillary tube 210 and 220, initial and final sealing areas 230, the plurality of precipitation reservoirs 240, and the horizontal crystal-observation window 250.

FIG. 5a shows the top view of a capillary plate for 6×16 counter-diffusion experiments.

FIG. 5b shows the corresponding side view of the capillary plate shown in FIG. 5a.

FIG. 5a and FIG. 5b illustrate an assembled capillary plate, the flexible fused silica capillary tube 100, the optical registry markers 205, the rectangular cuboid shaped support frame 200, the two ends of the flexible fused silica capillary tube 210 and 220, initial and final sealing areas 230, the plurality of precipitation reservoirs 240, and the horizontal crystal-observation window 250.

FIG. 6 illustrates the top view of a capillary plate for 2×24 counter-diffusion experiments. FIG. 6 illustrates an assembled capillary plate, the flexible fused silica capillary tube in column-orientation 100, the optical registry markers 205, the rectangular cuboid shaped support frame 200, the two ends of the flexible fused silica capillary tube 210 and 220, initial and final sealing areas 230, the plurality of precipitation reservoirs 240, and the horizontal crystal-observation window 250.

FIG. 7a shows the top view of a high density capillary plate for 3×22 counter-diffusion experiments. FIG. 7a illustrates an assembled capillary plate, the flexible fused silica capillary tube 100, the optical registry markers 205, the rectangular cuboid shaped support frame 200, the two ends of the flexible fused silica capillary tube 210 and 220, initial and final sealing areas 230, the plurality of precipitation diamond shaped reservoirs in a zigzag orientation 240, and the horizontal crystal-observation window 250.

FIG. 7b shows the top view of a capillary plate for 16×3 counter-diffusion experiments for 16 protein samples. FIG. 7b illustrates an assembled capillary plate, the 16 flexible fused silica capillary tubes 100, the optical registry markers 205, the rectangular cuboid shaped support frame 200, the 2×16 ends of the flexible fused silica capillary tubes 210 and 220, initial and final sealing areas 230, the plurality of precipitation reservoirs 240, and the horizontal crystal-observation window 250.

FIG. 7c shows the top view of a high density capillary plate for 2 protein samples each for 3×16 counter-diffusion experiments. This configuration would allow two independent protein crystallization experiments to be performed using each precipitation wells for 2 experiments. FIG. 7c illustrates an assembled capillary plate, the two flexible fused silica capillary tubes 100 and 101, the optical registry markers 205, the rectangular cuboid shaped support frame 200, the four ends of the flexible fused silica capillary tubes 210, 211, 220 and 221, initial and final sealing areas 230, the plurality of precipitation reservoirs 240, and the horizontal crystal-observation window 250.

FIG. 7d shows the top view of a modified capillary plate for crystallizing macromolecules using the batch under-oil technique. This configuration requires a premixing the protein sample and the precipitation solution prior loading this mixture as nano-drops into the capillary. FIG. 7d illustrates such a modified capillary plate, the flexible fused silica capillary tube 100, the optical registry markers 205, the rectangular cuboid shaped support frame 200, the two ends of the flexible fused silica capillary tubes 210 and 220, and the horizontal crystal-observation window 250. The enlargement of FIG. 7d illustrates a successful batch under-oil crystallization experiment with protein crystals 700, in nano-drops 310 spaced between water immiscible liquid 320.

FIG. 7e shows the top view of a capillary plate for 3×16 counter-diffusion experiments, whereby the capillary tube inlet 210 and outlet 220 starts inside of the rectangular support frame. FIG. 7e illustrates an assembled capillary plate, the flexible fused silica capillary tube 100, the optical registry markers 205, the rectangular cuboid shaped support frame 200, the two ends of the flexible fused silica capillary tube 210 and 220 starting and ending inside of a precipitation well, initial and final sealing areas 230, the plurality of precipitation reservoirs 240, and the horizontal crystal-observation window 250.

FIG. 7f shows the top view and two enlarged side views of a micro fluidic capillary plate for 3×16 counter-diffusion experiments, whereby the continuous polymer channel inlet 215 and outlet 225 starts inside of the rectangular support frame 200. FIG. 7f illustrates an assembled micro fluidic capillary plate, the single continuous micro fluidic polymer channel 105 is imbedded in a polymer waver 201, the two ends of the micro fluidic polymer channel 215 and 225 starting and ending inside of a precipitation well, initial and final sealing areas 230, the plurality of precipitation reservoirs 240 with a pierceable membrane on the bottom 106 of each well, the optical registry markers 205, the rectangular cuboid shaped support frame 200, and the horizontal crystal-observation window 250.

FIG. 8 shows a subsection of the top and corresponding side view of an unused capillary plate with an unfilled flexible fused silica capillary tube. FIG. 8 illustrates an assembled empty capillary plate, the empty flexible fused silica capillary tube 100, the optical registry markers 205, the rectangular cuboid shaped support frame 200, the two ends of the flexible fused silica capillary tube 210 and 220, initial and final sealing areas 230, the plurality of precipitation reservoirs 240, and the horizontal crystal-observation window 250.

FIG. 9 shows a subsection of the top and corresponding side view of a capillary plate with the flexible fused silica capillary tube filled with protein solution 300. FIG. 9 illustrates an assembled capillary plate, the flexible fused silica capillary tube 100, the optical registry markers 205, protein solution 300, the rectangular cuboid shaped support frame 200, the two ends of the flexible fused silica capillary tube 210 and 220, initial and final sealing areas 230, the plurality of precipitation reservoirs 240, and the horizontal crystal-observation window 250.

FIG. 10 shows a subsection of the top and corresponding side view of a capillary plate with the flexible fused silica capillary tube filled with protein solution 300 and with cleaved capillary tubes 400 in the sealing areas 230 creating capillary tube segments filled with protein solution which are sealed in both ends. FIG. 10 illustrates an assembled capillary plate, the flexible fused silica capillary tube 100, protein solution 300, the cleaved capillary tubes in the sealing areas 400, the optical registry markers 205, the rectangular cuboid shaped support frame 200, the two ends of the flexible fused silica capillary tube 210 and 220, initial and final sealing areas 230, the plurality of precipitation reservoirs 240, and the horizontal crystal-observation window 250.

FIG. 11 shows a subsection of the top and corresponding side view of a capillary plate with the flexible fused silica capillary tube filled with protein solution 300, with cleaved capillary tubes 400 in the sealing areas 230 and with cleaved capillary tubes 500 in the precipitation wells 240, starting the experiments by counter-diffusion of the precipitation solution 600 into the capillary tubes 100. FIG. 11 illustrates an assembled capillary plate, the flexible fused silica capillary tube 100, protein solution 300, the cleaved capillary tubes in the sealing areas 400, the cleaved capillary tubes in the precipitation wells 500, sealing tape for minimizing the loss of the reservoir solutions due to evaporation 260, the rectangular cuboid shaped support frame 200, the optical registry markers 205, the two ends of the flexible fused silica capillary tube 210 and 220, initial and final sealing areas 230, the plurality of precipitation reservoirs 240, and the horizontal crystal-observation window 250.

FIG. 12 shows a subsection of the top and corresponding side view of an ongoing counter-diffusion crystallization experiment using a capillary plate. The enlargement shows a successful counter-diffusion crystallization experiment with protein crystals 700 in a capillary tube 100. FIG. 12 illustrates a subsection of a capillary plate with the flexible fused silica capillary tube 100, the rectangular cuboid shaped support frame 200, protein solution 300, the cleaved capillary tubes in the sealing areas 400 and in the precipitation wells 500, the optical registry markers 205, reservoir sealing tape 260, counter-diffusion of the precipitation solution 600 into the capillary tubes 100, initial and final sealing areas 230, the plurality of precipitation reservoirs 240, and the horizontal crystal-observation window 250.

FIG. 13 shows a subsection side view of a capillary plate. This capillary plate is vertically mounted in an X-ray diffractometer on a motorized x-/y-/z-axis plate stage 840 while the counter-diffusion crystallization experiment 600 is still ongoing. The X-ray beam 800 is positioned at an in-situ grown protein crystal 700. The protein crystal diffracts in-situ the X-ray beam and the resulting reflections 810 will produce distinct spots on the detector 820. For an entire in-situ analysis of a protein crystal by X-ray diffraction the motorized x-/y-/z-axis plate stage 840 will rotate the protein crystal around its axis φ 830. FIG. 13 illustrates a subsection of a capillary plate when vertically mounted in X-ray diffractometer, with the flexible fused silica capillary tube 100, the optical registry markers 205, ongoing counter-diffusion of the precipitation solution into the capillary tubes 600, the protein crystal 700, the incoming X-ray beam 800, the reflected X-ray beam 810, the detector 820, the motorized x-/y-/z/φ-axis plate stage 830+840, the cleaved capillary tubes in the sealing areas 400 and in the precipitation wells 500, reservoir sealing tape 260, initial and final sealing areas 230, and the precipitation reservoirs 240.

FIG. 14 an alternative technique of analyzing crystals in-situ by X-ray diffraction by removing the capillary from the capillary plate, sealing both ends of the capillary tubes with a capillary sealant and mounting the capillary onto a magnetic base compatible with standardized goniometer head. FIG. 14 illustrates a capillary 100 with protein crystals 700 sealed in both ends 850 mounted on a magnetic base 860, the incoming X-ray beam 800, the reflected X-ray beam 810, and the detector 820.

FIGS. 15, 16 and 17 illustrate the method of the crystal recognition software when automatically screening capillary images for protein crystals and assisting the researchers in their work. This crystal recognition software requires two images from the same location on the capillary plate. The images are preferably taken with the help of an automatic incubation and imaging system. The first, “time-zero” image, is taken immediately after starting the crystallization experiment, FIGS. 15a, 16a and 17a. A second image is taken after crystallization occurred, FIGS. 15b, 16b and 17b. The two images are aligned and the time-zero image is subtracted from the second image. The resulting image FIGS. 15c, 16c and 17c is converted to a black-and-white image to enhance the difference between the protein crystal 700 and the background FIGS. 15d, 16d and 17d. The crystal recognition software is programmed to recognize clusters of white pixels above a preset size threshold which indicates a protein crystal 700 grew at that location. The crystal recognition software flags that image file, marks the crystal position on the capillary and writes the x/y-coordinates relative to the optical registry markers 205, in a computer file.

FIG. 18 shows some possible geometrical shapes to be used as optical positional markers (also denoted “optical registry markers”) useful in the context of the present invention. The optical positional markers consist out of a very fine shape with a high contrast to its background. An optical registry marker can be one or more of very small apertures or a crosshair shaped slit in the support frame, any fine shape as part of the support frame mold, a small mirror or a label with an easy to centre print on it e.g. a crosshair (205a˜205i).

### DETAILED DESCRIPTION OF THE INVENTION

The invention will be described in more details with reference to the accompanying drawings, in which some, but not all possibilities of the invention are shown. The invention may be assembled in many different forms and should not be construed as limited to the constructs set forth herein. Like numbers refer to like elements throughout.

The invention relates to a capillary plate that is particularly useful for crystallizing biological macromolecules for X-ray diffraction analysis. Biological macromolecules include proteins, nucleic acid and viruses. The capillary plate provides a crystallization technique that incorporates all key crystallization steps in a single device. All steps are performed in-situ so it is not necessary for the researcher to manually manipulate the crystals. The capillary plate is designed for high-throughput crystallography and all operations can be performed under fully automated conditions.

A unique aspect of this invention is a one-time loading step of the sample of a solution of a biological molecule, e.g. a protein solution, into a long capillary or polymer channel (together “capillary tube”) which provided reaction compartments for a multitude of crystallization experiments. After loading the protein, the filled capillary or polymer channel is partitioned into a plurality of identical short isolated counter diffusion crystallization experiments by blocking the fluid communication between the individual segments. And to start the counter diffusion crystallization experiment each protein filled capillary or polymer channel segment is opened towards a compartment, e.g. a well, containing a second liquid (i.e. the precipitation solution).

The capillary plate usually comprises of the following elements: a support frame which is preferably rectangular, capillary tubes, preferably one continuous capillary tube, a plurality of units of first and second sealing areas precipitation reservoir, and inspection area, and a plurality of optical positional reverence points, referred to as optical registry markers.

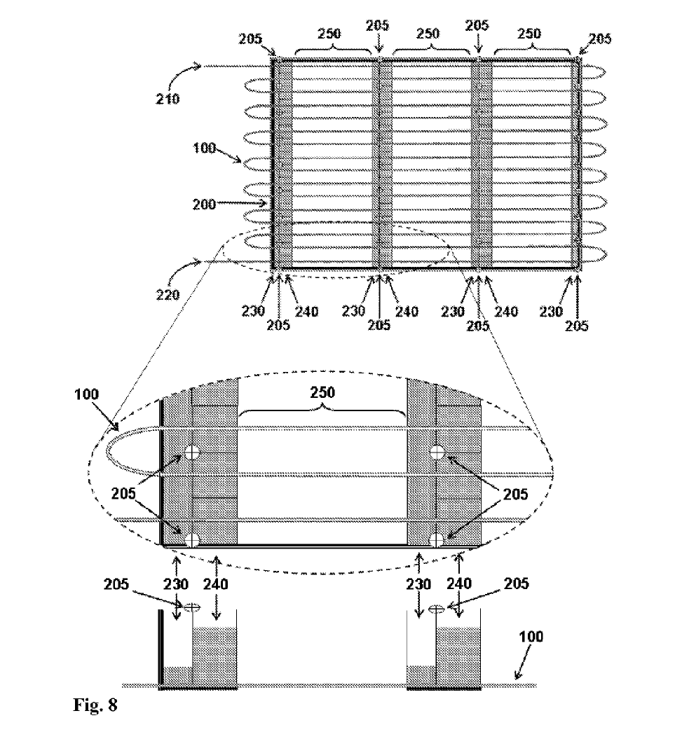

One unit (hereinafter also referred to as “crystallization zone”) equals to one counter diffusion crystallization experiment. A crystallization zone consists out of four building blocks: (a) an initial sealing area (FIG. 8, 230); (b) a precipitation reservoir well (FIG. 8, 240); (c) a preferably horizontal inspection area (FIG. 8, 250); and (d) a final sealing area (FIG. 8, 230).

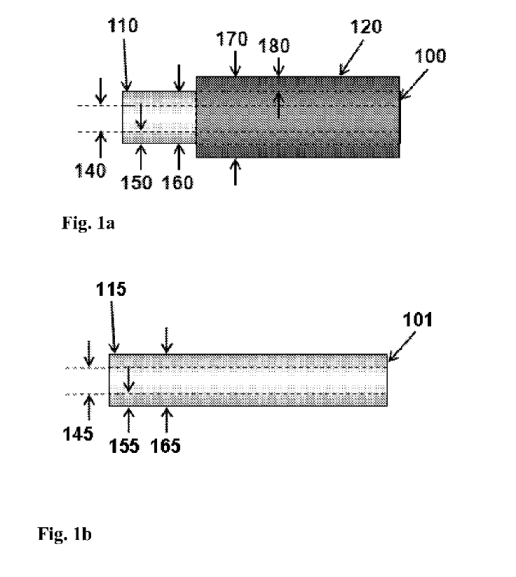

The continuous capillary tube (FIG. 1a, 100; FIG. 1b, 101) used in this invention passes through the four building blocks of the crystallization zone.

The continuous capillary tube (FIG. 1a, 100; FIG. 1b, 101) used in this invention passes through a plurality of crystallization zones.

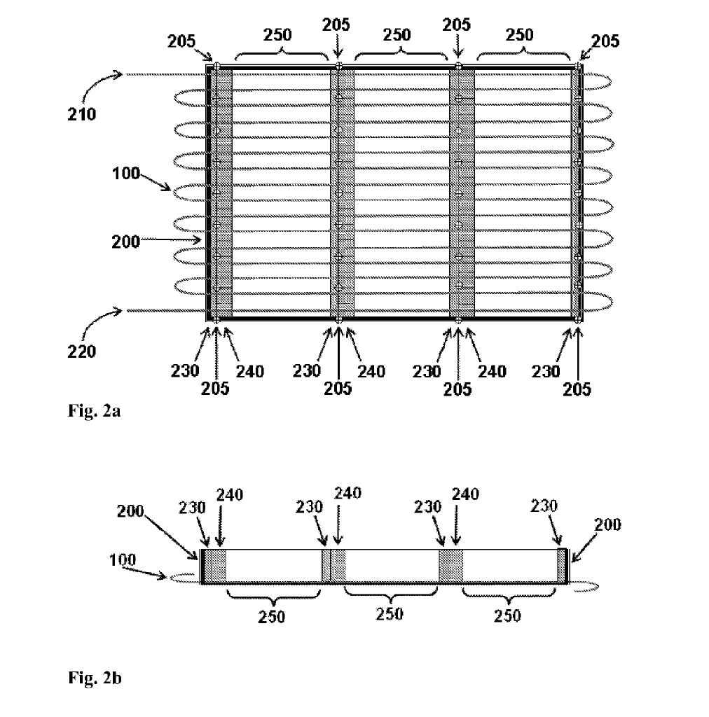

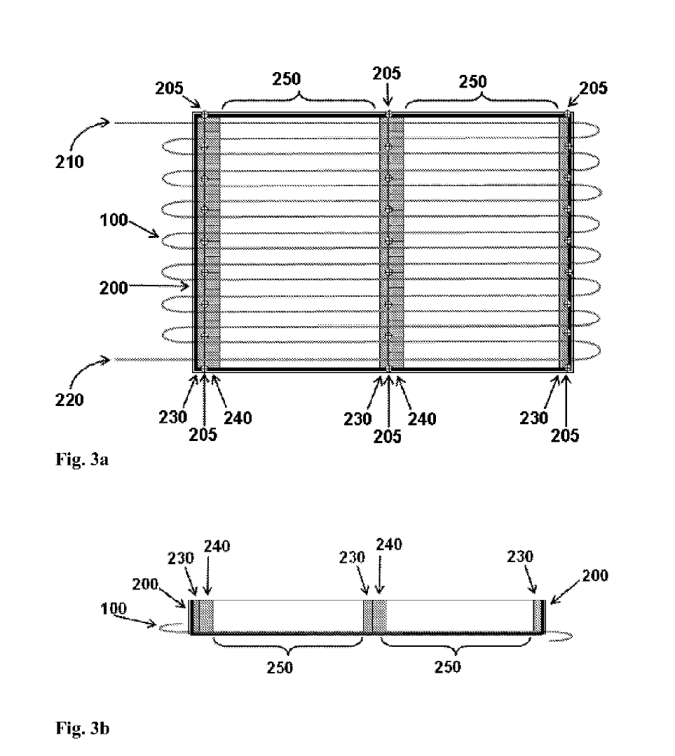

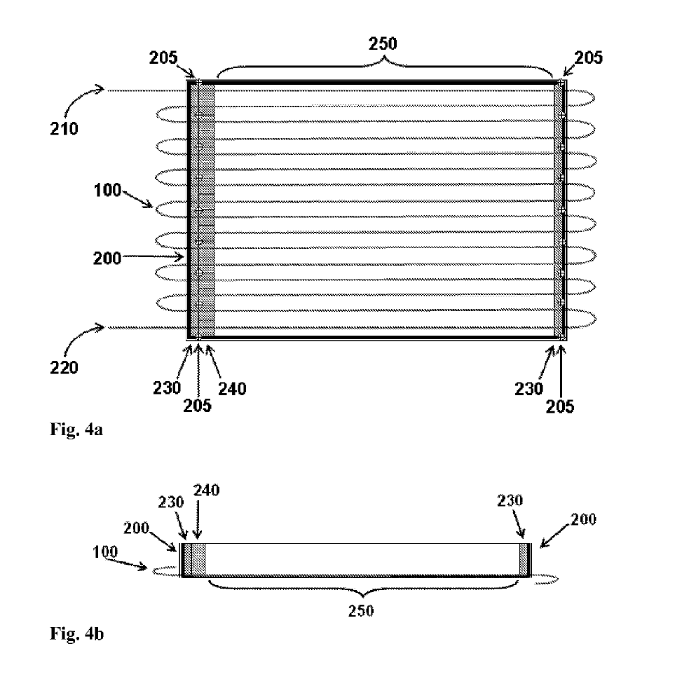

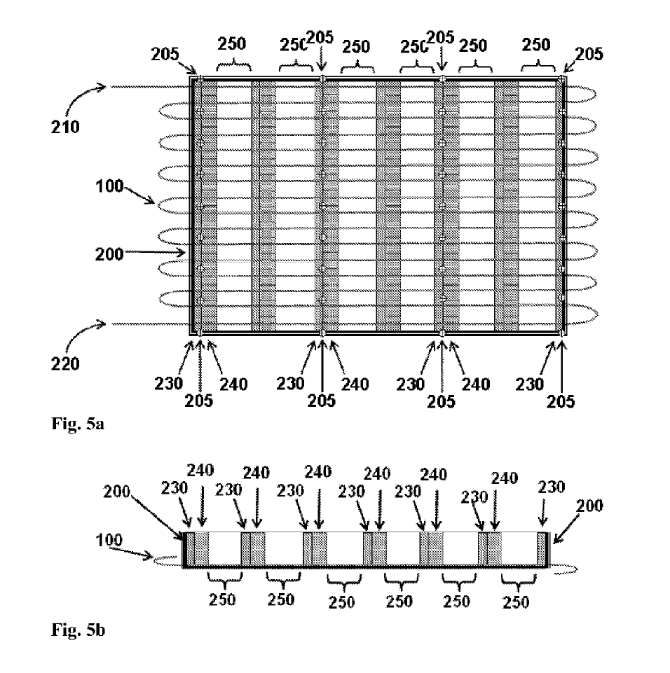

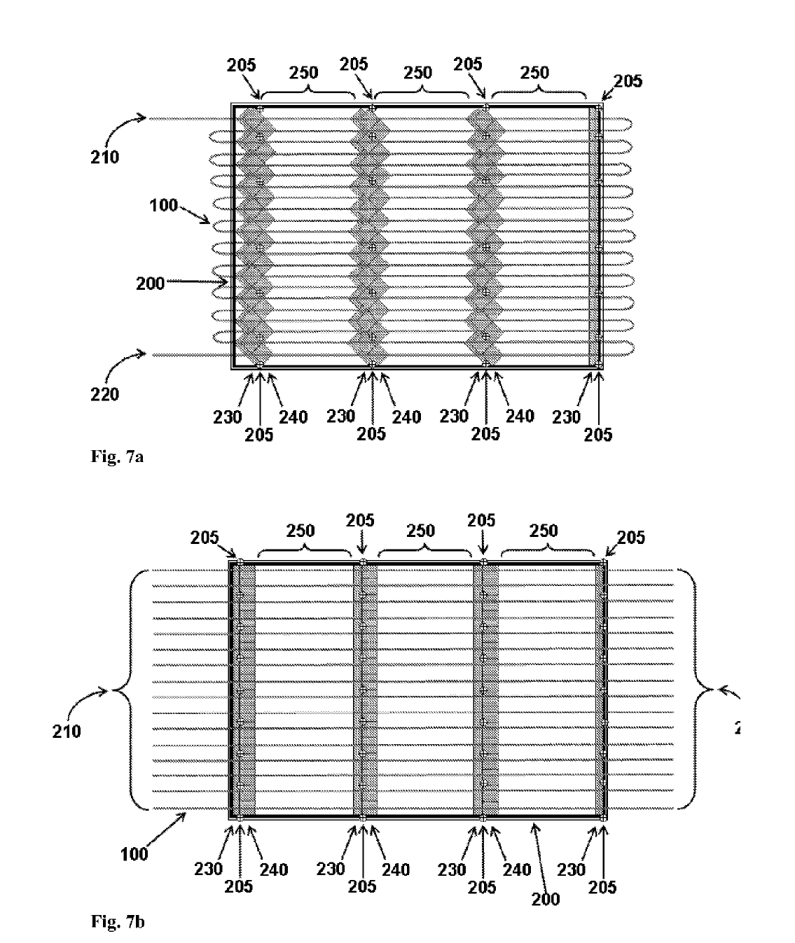

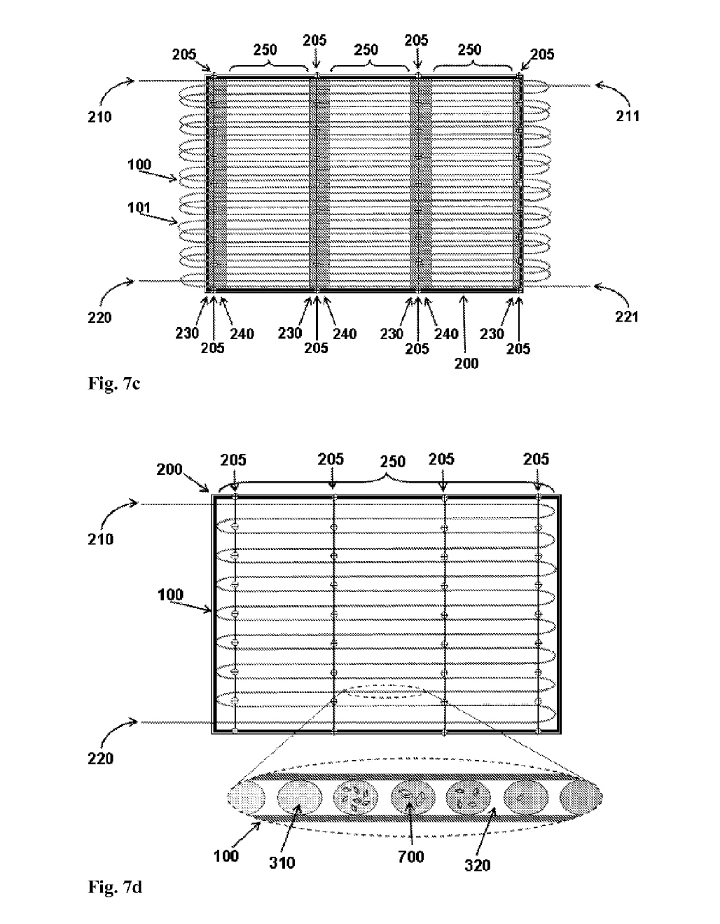

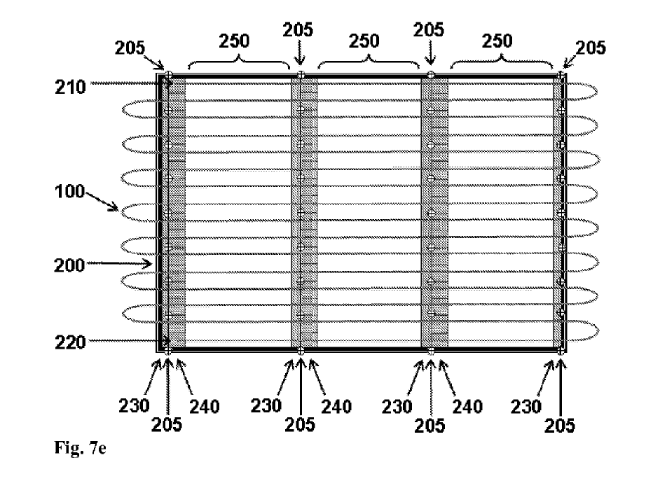

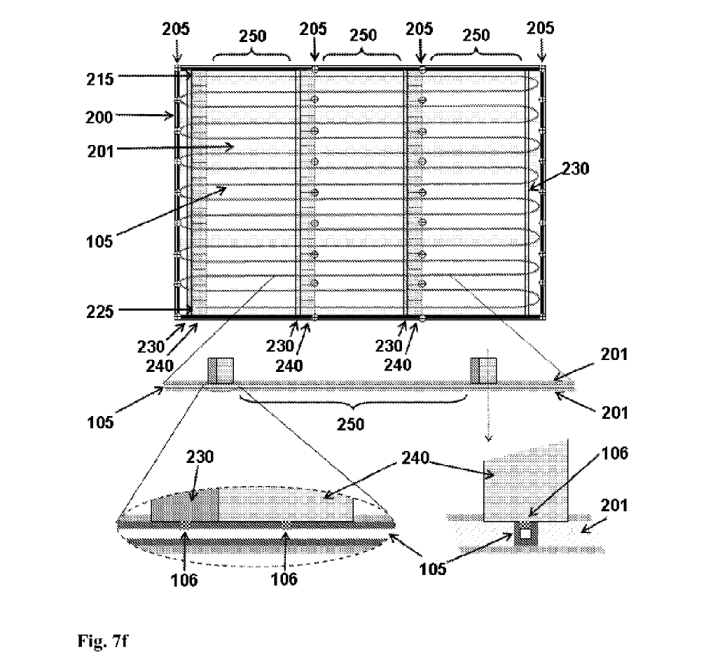

The numbers of capillary tubes passing through the same crystallization zones are preferably ranging from one to two capillary tubes (FIG. 7c), more preferred one capillary tube is present (FIG. 2a).

The inlet and outlet of the capillary tube can start inside or outside of the rectangular support frame, preferably outside of the support frame (FIG. 2a). When the two ends start inside of the rectangular support frame, it preferably starts inside of the first and the last precipitation reservoir (FIG. 7e).

The length of one crystallization zone ranges preferably from 1 mm to 100 mm, more preferably 10 mm to 40 mm. The length of a crystallization zone will determine the number of counter diffusion crystallization experiment that can be performed on one capillary plate. The number of crystallization zones per capillary plate is usually from 8 to 1536 zones, preferably 16 to 96 zones.

The capillary plate can also consist of one capillary tube (FIG. 7d) positioned in a serpentine pattern inside of the support frame with one continuous horizontal inspection area (herein after also referred to as “crystal-observation window”) (250). This plate configuration allows the crystallization of macromolecules using the batch under-oil technique in the nanoliter scale.

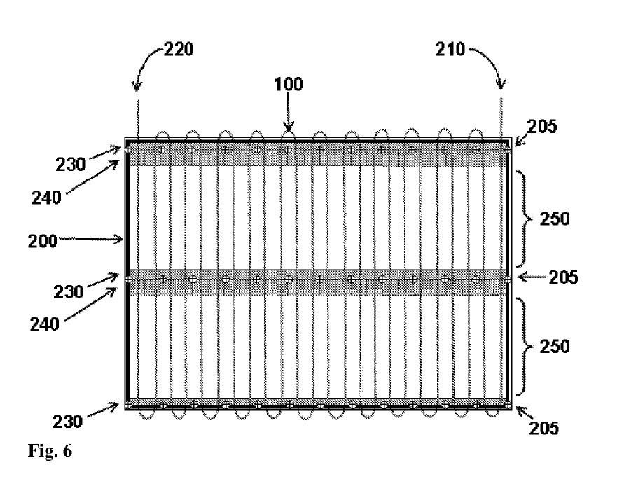

The orientation of the crystallization zones on the capillary plate can be left-right (e.g. FIG. 2a) or it can be back to front (FIG. 6), preferably the orientation is from left to right.

The number of capillary tubes used on a capillary plate preferably ranges from one continuous tube to 48 individual tubes, more preferably one to two continuous capillary tubes.

The capillary tube (FIG. 1a, 100) used in this invention is preferably a glass tube synthesized from highly purified silica, which is coated externally with a protective polymer to yield a transparent and highly flexible tube.

The glass tube (FIG. 1a, 110) can be made of, e.g. natural quartz or borosilicate glass, preferably highly purified silica.

The capillary tube used in this invention (FIG. 1a, 100; FIG. 1b, 101), is defined as “any tube with a small internal diameter” such that capillary forces can apply, and is constructed of an amorphous material that is suitable for X-ray diffraction.

The inner surface of the flexible capillary tubing can be modified to alter its inner surface property by immobilizing an anchor protein, by blocking, activating, deactivating, rinsing or by chemically derivatizing the glass surface.

The solvents for rinsing the inner glass surface of the capillary tubing may be water, methanol, ethanol, propanol, butanol, acetone, acetonitrile, ethyl acetate, pentane, hexane, dichloromethane, and combinations thereof.

The acids and caustics for activating the inner glass surface of the capillary tubing are hydrofluoric acid, nitric acid, sulfuric acid, sodium-, potassium-hydroxide, and combinations thereof.

The chemical agents suitable for derivatizing the inner glass surface of the capillary tubing are Trimethylchlorosilane (TMCS), Dimethyldichlorosilane (DMDCS), Triethylchlorosilane (TESCl), Hexamethyldisilazane (HMDS), Triisopropylchlorosilane (TIPSCI), 1,3-Diphenyl-1,1,3,3-tetramethyldisilazane (DPTMDS), N,O-Bis(trimethylsilyl)acetamide (BSA), 1-(Trimethylsilyl) imidazole (TMSI), 1,1,3,3-Tetraphenyl-1,3-dimethyldisilazane (TPDMDS), N,O-Bis(trimethylsilyl)trifluoroacetamide (BSTFA), (3-Aminopropyl) triethoxysilane (APTES), glutaraldehyde and combinations thereof.

The polymer used for the external coating (FIG. 1a, 120) of a flexible fused silica capillary tubing is standard polyimide, Teflon®, high temperature polyimide, acrylate, UV-transparent fluoropolymer, fluorinated acrylate, silicone, polyethylene, polypropylene, acrylic poly(methyl methacrylate), polystyrene, polyethylene terephthalate (PET), polycarbonate polymers, CR-39, copolymers of styrene or Nylon® and combinations thereof, preferably standard polyimide and Teflon®.

The capillary tube are filled, rinsed and emptied with liquids preferably by either applying a positive pressure on the inlet side or a vacuum on the outlet side.

The capillary tube (FIG. 1b, 115) used in this invention can also be made entirely out of a polymer. The polymer capillary tube can be made, e.g. of Teflon®, standard polyimide, high temperature polyimide, acrylate, UV-transparent fluoropolymer, fluorinated acrylate, silicone, polyethylene, polypropylene, acrylic poly(methyl methacrylate), polystyrene, polyethylene terephthalate (PET), polycarbonate polymers, CR-39, copolymers of styrene and Nylon® and combinations thereof, preferably Teflon®.

Even though the external protective polymer coating (FIG. 1a, 120) makes the capillary tube (FIG. 1a, 100) very strong and abrasion resistant, it easily can be cleaved at a precise location by nicking this protective polymer layer, e.g. with a sharp scalpel.

The length of the capillary tube (FIG. 1, 100) is preferably from 0.05 m to 20 m, more preferably from 2 m to 6 m.

The inside diameter of the capillary tube (FIG. 1a, 140) is preferably from 0.002 mm to 0.7 mm, e.g. 0.1 mm.

The wall thickness of the inner capillary tube (FIG. 1a, 150) is preferably from 0.01 mm to 0.16 mm, more preferably 0.02 mm.

The external diameter of the fused silica capillary tube (FIG. 1a, 160) is preferably from 0.066 mm to 0.82 mm, e.g. 0.14 mm.

The coating thickness per side of the capillary tube, in particular if present in the form of a flexible fused silica tube (FIG. 1a, 180), is preferably from 0.01 mm to 0.03 mm, e.g. 0.012 mm.

The external diameter of such a tube (FIG. 1a, 170) is typically from 0.09 mm to 0.85 mm, e.g. 0.164 mm.

The inside diameter of the capillary tube (FIG. 1a, 145) is usually from 0.002 mm to 0.7 mm, e.g. 0.1 mm.

The wall thickness of the capillary tube (FIG. 1a, 155) may be, e.g. from 0.002 mm to 0.2 mm, preferably 0.02 mm.

The external diameter of the capillary tube (FIG. 1a, 165) is preferably from 0.05 mm to 0.9 mm, e.g. 0.14 mm.

The sealing area (FIG. 8, 230) is typically a 1mm-5 mm wide region having a depth of e.g. 2 mm -5 mm, preferably 3 mm in widths and 3 mm in depths, containing a water immiscible sealing paste, preferably selected from the group consisting of silicone-based vacuum grease, petroleum-based vacuum grease, petroleum jelly, Vaseline®, “Melkfett”, paraffin, wax, tar, clay, beeswax, dental wax, latex, plastic plugs, agarose, fluids with a thixotropic property and combinations thereof, preferably silicone-based high vacuum grease.

The precipitation reservoirs (FIG. 8, 240) usually have a round, rectangular or other shape, a diameter from, e.g. 0.1 mm to 10 mm, preferably from 4 mm to 5 mm, and usually a volume of from 0.005 ml to 0.5 ml, preferably from 0.02 ml to 0.15 ml.

According to a preferred embodiment of the invention, the capillary plate has a plurality of optical registry markers (205). The optical registry markers can be a fine shape, a figure, an outline, a silhouette, a profile or a contour with e.g. a high contrast to its background. An optical registry marker can be one or more small holes, a small aperture or a crosshair shaped slit in the support frame, any fine shape as part of the support frame mold, a small mirror or a label with a crosshair, target or partial squares. The size of an optical registry marker is typically between 0.001 mm and 0.1 mm, preferably between 0.005 mm and 0.025 mm.

The optical registry makers make it possible to determine relative x-/y-coordinates from any position on the capillary plate. The x-/y-stage on an X-ray diffractometer may also be equipped with a system recognizing the optical registry makers and determine the exact positions. This position fine tuning is of benefit when transferring the x-/y-coordinates from a crystal derived from the crystal recognition software to the x-/y-stage on the X-ray diffractometer.

The horizontal crystal-observation window (FIG. 8, 250) is a visual open area, where the capillary tubes can be examined visually or optically, preferably with the aid of a microscope. To keep the capillary tubes in the horizontal crystal-observation window straight, the capillary tubes can be supported by a clear plastic foil.

In a preferred embodiment of the capillary plate of the invention, the capillary tube (FIG. 8, 100), leads from the outside to the inside of the rectangular support frame (FIG. 8, 200) through a capillary passageway. Inside of the frame, the capillary tube passes as a continuous tube through a plurality of crystallization zones. In a crystallization zone, the capillary tube passes first through the initial sealing area (FIG. 8, 230) then enters the precipitation reservoir well (FIG. 8, 240) through its sidewall. After passing through the precipitation reservoir, typically on or beneath its bottom area of the well, the capillary tube exits the precipitation reservoir by passing through the opposite sidewall. The path of the capillary tube continues through the inspection area (FIG. 8, 250) and is ending its cycle by passing through the second sealing area (FIG. 8, 230). The second sealing area can be identical with the first sealing area of the next crystallization zone. The capillary tube passes sequentially through all the crystallization zones in the first row as just described. At the end of the row the capillary tube makes a 180° U-turn, passing in reverse through the crystallization zones in the second row. At the end of the second row, the capillary tube makes another 180° U-turn, passing through the crystallization zones in the third row. The capillary tube's serpentine or meander-like pattern is repeated until the last row of the capillary plate has been reached. The capillary tube exits the rectangular support frame through a second capillary passageway. Arbitrarily one end of the capillary tube is designated as the “proximal end” or “inlet” (FIG. 8, 210), the other end is designated as “distal end” or “outlet” (FIG. 8, 220). The capillary tube used in this embodiment of the invention is one single continuous flexible tube from start to end, passing through a plurality of crystallization zones, whereby the inside of the capillary tube is initially not in fluid communication with the precipitation reservoirs. The precipitation reservoirs in the capillary plate of the present invention preferably contain a precipitating solution, cryoprotectant solution or a strong X-ray scattering heavy metal ion component.

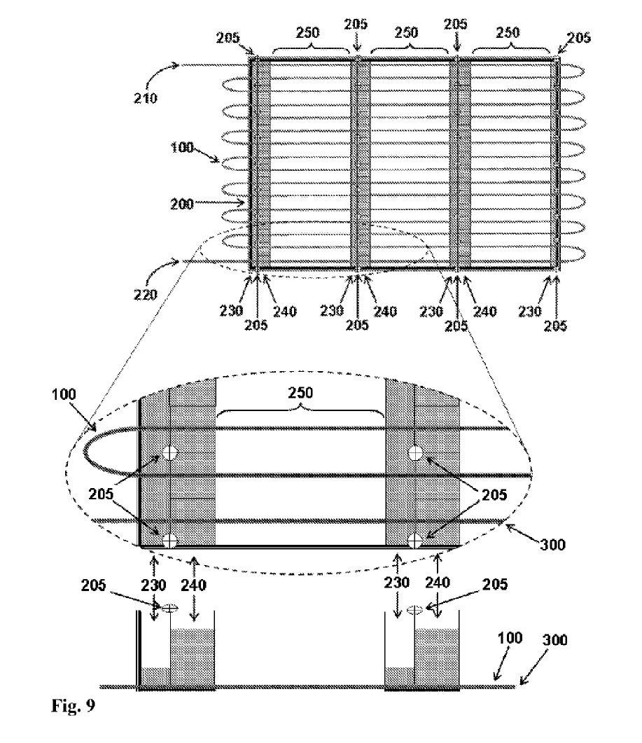

The biological molecule, preferably protein sample is placed in a small sample tube. The proximal end (inlet) of the capillary tube is positioned into this sample tube so that the capillary tip contacts the sample. The sample enters into the capillary by capillary force. This protein loading step can be expedited by applying either a positive pressure on the sample tube or a vacuum on the outlet side (distal end) of the capillary tube. After the entire capillary tube is filled with the sample solution, the sample tube is removed from the inlet (proximal end) of the capillary tube (FIG. 9).

At this state of the experiment, the capillary plate still contains one continuous capillary tube filled with sample solution. The polymer coating and the glass wall of the protein filled capillary tube separates the protein sample from the reservoir solutions.

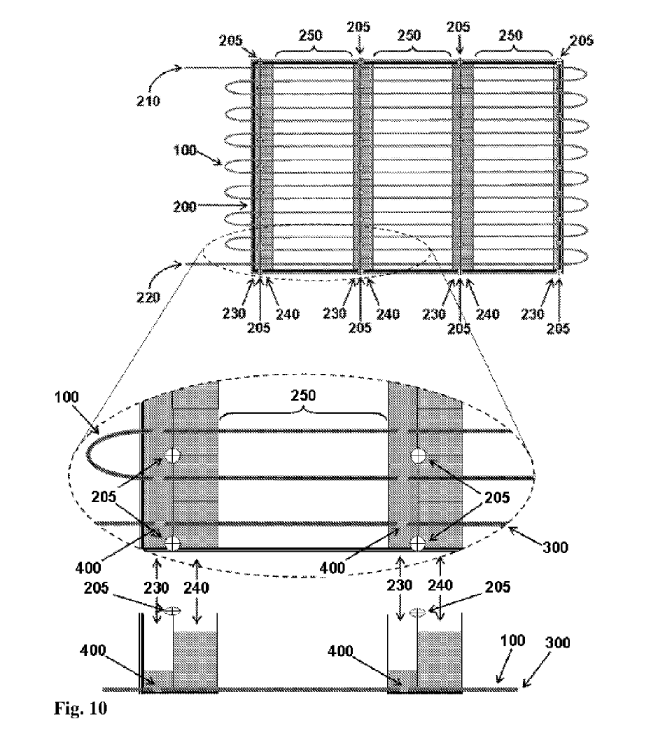

By using a means for cutting the capillary tube, e.g. a scalpel, the capillary tube is cleaved in all (first and second) sealing areas (FIG. 10, 400). The newly created capillary ends are moved away from each other into the sealing paste, so the fluid communication is permanently blocked. This step creates multiple identical capillary tubes containing the sample solution and being sealed at both ends. The polymer coating and the glass wall of the capillary tube filled with sample still separates the sample solutions from the reservoir solutions.

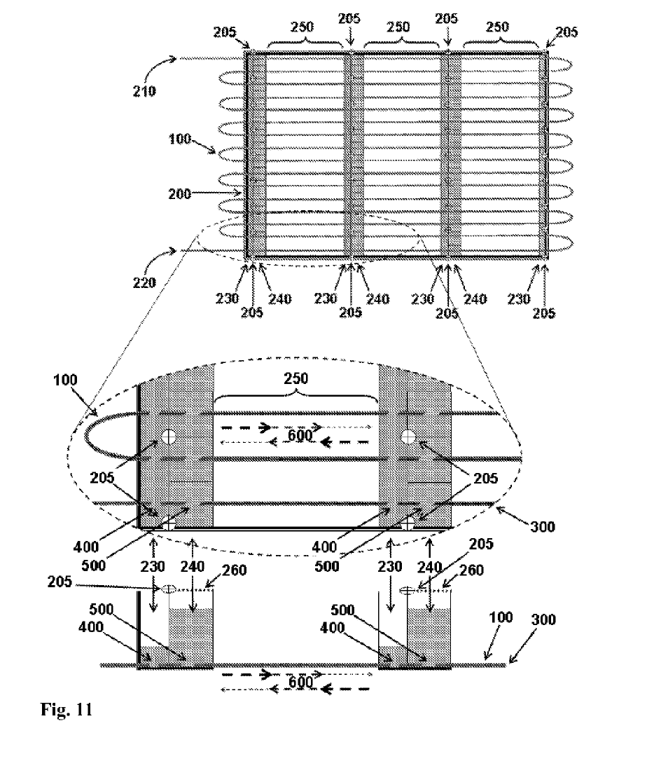

In a second step, all capillary tube segments are cleaved with a cutting device such as a scalpel in the reservoir beneath the liquid level of the reservoir solutions (FIG. 11, 500). The sample solution is now in contact with the second liquid, preferably a precipitation solution, in the precipitation reservoir(s) and starts the crystallization experiment. Especially in the case of a protein solution as the sample solution, the protein solution and the reservoir solution diffuse against each other and a wave of supersaturated protein is formed and moves through each capillary. Protein crystals begin to form as the wave of supersaturated protein moves through each of the capillaries.

As an alternative of cutting the capillary one-by-one with a scalpel, this step can be expedited with a comb shaped cutting device for cleaving all capillary tubes in one single step.

As an alternative of cutting the capillary in the sealing areas, the capillary tube can be also compressed or heat sealed by melting the tube material in order to permanently block any fluid communication between the short capillary tube segments.

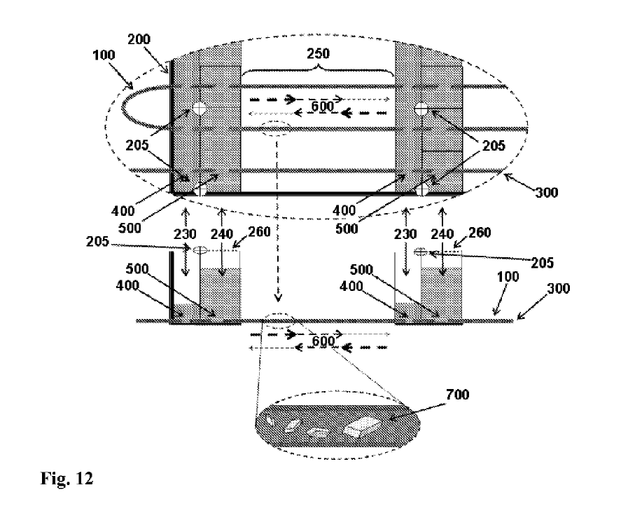

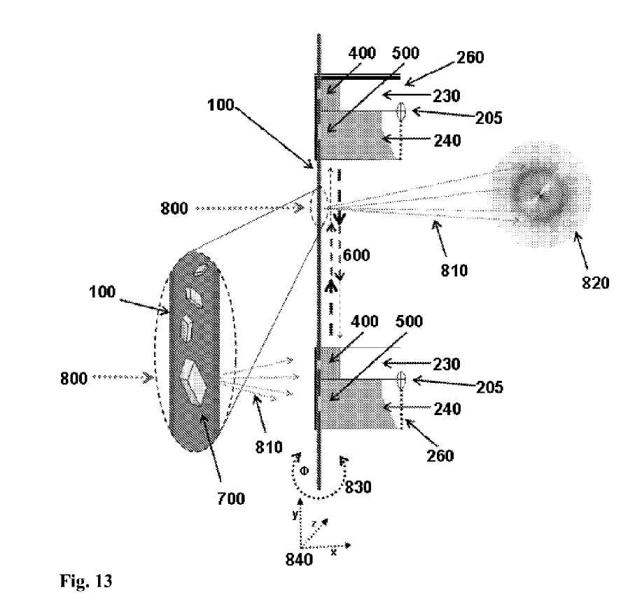

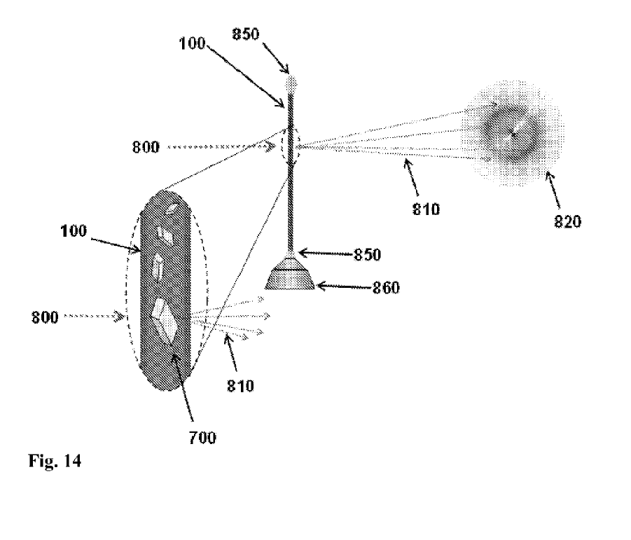

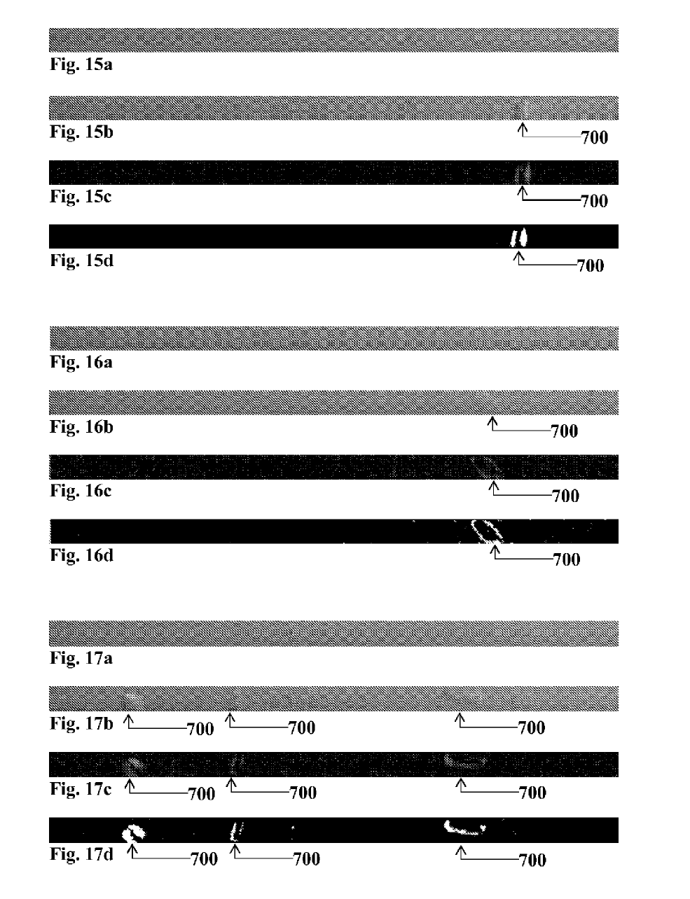

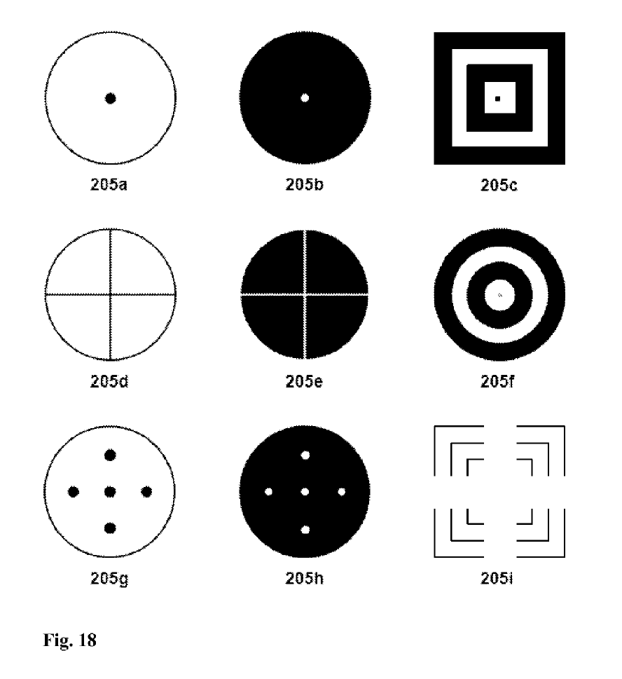

During the interface diffusion experiment, a constant moving precipitant gradient is formed along the length of the capillary tube (FIG. 12, 600). Parallel to this gradient moves a protein supersaturation zone along the length of the capillary tube (FIG. 12, 600), which leads to crystal growth (FIG. 12, 700). Each capillary plate will greatly improve the chances of obtaining crystals that are suitable for X-ray analysis. This invention is a major step forward in achieving the goal of solving the structures for novel biological molecules, in particular proteins, by making feasible a high throughput crystallization screening in (preferably glass) capillaries by the counter-diffusion method combined with in situ X-ray diffraction data collection of (in particular protein) crystals without manual manipulation.

Cleaving the capillary tube beneath the liquid level of the reservoir solution ensures an air bubble free interface between the protein- and the reservoir solution. An air bubble between the protein- and the reservoir solution would hinder the diffusion to take place and could prevent the crystallization experiment to start.

Preferably, all precipitation reservoirs are sealed with a sealing tape to reduce evaporation of the reservoir solutions (FIG. 11, 260).

In a preferred embodiment of this capillary plate invention using the batch under-oil crystallization technique, the capillary tube (FIG. 7d, 100) leads from the outside to the inside of the rectangular support frame (FIG. 7d, 200) through a capillary passageway. The capillary tube is meandering as a continuous tube from one frame side to the other of the rectangular support frame always making a 180° U-turn. The capillary tube's serpentine pattern is repeated until the last row of the capillary plate has been reached. The capillary tube exits the rectangular support frame through a second capillary passageway. One end of the capillary tube is designated as the “proximal end” or “inlet” (FIG. 7d, 210), the other end is designated as the “distal end” or “outlet” (FIG. 7d, 220).

By using an external device the sample, preferably protein, solution is mixed with a variety of precipitation solutions in various volume ratios and loaded as small drops sandwiched in between a water immiscible liquid spacer. Immediately after the loading process the inlet and the outlet of the capillary tube is sealed with a capillary sealant. This plate configuration allows the crystallization of macromolecules using the batch under-oil technique, preferably in a volume range of 0.5 nl to 5 nl.

The horizontal orientation of the capillary plate allows a visual or optical inspection of the capillary tubes in the inspection areas (FIG. 12, 250), preferably by using an optical stereomicroscope to select crystals for X-ray diffraction without disturbing the ongoing crystallization experiment.

The capillary plate is preferably designed for use in combination with commercial incubation and imaging systems. Such systems allow the capillary plate to be incubated at a set temperature and the capillaries are automatically imaged several times over the duration of the experiment. The standardized geometry of the capillary plate simplifies the adjustment of the image focus. The clearly defined straight border and the high contrast between the capillaries and its background allow a fully automatic focusing. Preferably, the incubation and imaging system are also programmed to take images from all optical registry markers at the same time.

By using the capillary plate of the present invention, images from a unit of first and second sealing areas, precipitation reservoir and inspection areas, preferably are taken with the help of an automatic incubation and imaging system from the inspection area(s), allow a novel approach for the application of a crystal recognition software by electronically subtracting a first image (or the image information of said first image) taken at time zero (or a generally a first time point) from the same image (i.e. a second image or the image information thereof) taken from the same position at a later second (usually the current) time point. The images from the optical registry markers and the straight profile of the capillaries allow the software to perfectly overlay electronically the two capillary images for the subtraction. The two images are aligned and the time-zero image is subtracted from a current image. The resulting image is preferably converted to a black-and-white image to enhance the difference between the crystal of the biological molecule, e.g. a protein, and the background. In capillaries were no crystal growth occurred, the two images look identical and the subtracted image is black. In capillaries were crystal growth occurred, the two images look different at the position of the crystal and the image resulting from the subtraction does show a white spot at that location. The crystal recognition software is programmed to recognize clusters of white pixels above a preset threshold which indicates a crystal of the biological molecule, especially a protein. The crystal recognition software flags that image file and marks the position on the image. By making use of the position of the optical registry markers, the software computes the relative x/y-coordinates of the crystal. The position of the optical registry markers and the relative x/y-coordinate of the crystals will be used when analyzing the crystals by in situ X-ray diffraction.

For analyzing crystals in situ by X-ray diffraction, the capillary plate is preferably mounted on a motorized x-/y-/z-axis plate stage. The motorized x-/y-/z-axis plate stage may be equipped with a device recognizing the position of optical registry markers. The device for recognizing the optical registry marker can be a camera or a laser beam/light detector. The image from the camera is analyzed by software for recognizing the optical registry markers. The laser beam/light detector recognizes optical registry marker such as a small mirror, a fine hole, a crosshair or any small opening in the capillary plate. In either case the x-/y-positions from the optical registry marker on the X-ray diffraction stage is determined. The x-/y-coordinates of the crystals, determined by the crystal recognition software are normalized to the position of the optical registry markers. The motorized x-/y-/z-axis plate stage can therefore move the capillary plate so that a target crystal is placed in an X-ray beam for data collection. After applying an optional stream of cryogenic gas to the capillary tube the motorized x-/y-/z-axis plate stage will rotate the crystal around its own axis φ (830) for X-ray diffraction data collection.

For analyzing crystals in situ by X-ray diffraction, a single capillary tube containing the crystal, in particular a protein crystal, is manually removed from the crystallization plate. Immediately both ends of the capillary tube are sealed with a capillary sealant. The sealed capillary tube segment is mounted onto a magnetic base which fits the goniometer head in an X-ray diffractometer. After applying an optional stream of cryogenic gas to the capillary tube, the capillary tube with the crystal inside is rotated around its own axis in the X-ray beam and X-ray diffraction data are collected.

Summing up, the present invention provides a capillary plate, preferably for growing, cryoprotecting, incorporating scattering atoms, and analyzing macromolecular crystals in situ for direct macromolecular structure determination.

Furthermore, the present invention provides a method for the crystallization of biological molecules, preferably proteins, nucleic acids, viruses and combinations thereof, by using the interface diffusion (or counter diffusion) crystallization technique, preferably in glass capillaries fixed on a solid support, typically comprising the steps of:
preparing a capillary crystallization plate starts with conditioning or modifying the inner surface of the capillary tube by applying a procedure from the group consisting of washing, activating, deactivating, protein immobilization, blocking, derivatizing, and combinations thereof;
preparing a capillary crystallization plate by pre-loading the precipitation reservoirs with precipitating solutions, cryoprotectant solutions, and/or strong X-ray scattering heavy metal ion solutions;
loading the protein sample into the capillary tube by dipping one open end of the capillary tube into the protein solution. The capillary tube is filled with the protein solution by capillary force over the entire length. To expedite the protein loading step, positive pressure or vacuum can be applied to the capillary tube inlet or outlet, respectively;
cleaving the capillary tube in all sealing areas with a scalpel inside the sealing paste and moving the newly created capillary ends away from each other into the sealing paste. Blocking the fluid communication will create capillary tube segments filled with protein solution and sealed in both ends. As an alternative of physically separating the capillaries with a scalpel, compressing or sealing with heat the capillary tubes to permanently block any fluid communication with the result of creating capillary tube segments filled with protein solution which is sealed in both ends;
cleaving the capillary tube in the precipitation reservoir beneath the liquid surface level of the precipitating—or cryoprotectant solutions starts the interface diffusion crystallization experiment. This step brings the protein solution in the capillary tube in contact with the precipitating—or cryoprotectant solutions. The two last steps can be combined with a comb shaped cutting device for cleaving all capillary tubes simultaneously;
diffusing the precipitation solution or cryoprotectant solution into the capillary tubes;
sealing all precipitation reservoirs with sealing tape for minimizing the loss of the reservoir solutions due to evaporation;
growing crystals from macromolecules in the capillary tubes;
visually or optically tracking the crystallization experiment(s) by monitoring the capillary segments in the inspection area(s) using a microscope or by using an automatic incubator and imaging system;
visually or optically monitoring the crystallization experiment(s) for crystals. This is preferably assisted by automatic crystal recognition software;
selecting crystals for X-ray diffraction;
determining for each selected crystal the precise x-/y-coordinates for each selected crystal relative to the optical registry markers, preferably by using a crystal recognition software;
mounting the plate in an X-ray diffractometer on a motorized x-/y-/z-axis adaptor and normalizing the plate's position with the optical registry markers; and
analyzing the crystals in situ by X-ray diffraction by mounting the plate in an X-ray diffractometer on a motorized x-/y-/z-axis adaptor for moving the crystal in the X-ray beam and collecting X-ray data in situ. The crystal x-/y-positioning data relative to the optical registry markers derived from the crystal recognition software will assist in the precise positioning of the crystal in the X-ray beam by the motorized x-/y-/z-axis adaptor. Particularly at synchrotron sources
An alternative procedure of analyzing the crystals in-situ by X-ray diffraction is by removing a capillary tube segment containing the protein crystal from the crystallization plate and sealing both ends of the capillary tubes with a capillary sealant. The sealed capillary tube segment is mounted onto a goniometer head in an X-ray diffractometer and X-ray data are collected.

1-25. (canceled)

26. For use in the growth and analysis of macromolecular crystals, a glass capillary tube externally coated with a polymer.

27. A tube as claimed in claim 26 which is flexible and made from quartz glass,

28. A tube as claimed in claim 26 which is flexible and made from borosilicate glass.

29. A tube as claimed in claim 26 which is flexible and made from silica.

30. A tube as claimed in claim 26 wherein the coating is of a protective polymer selected from the group consisting of standard polyimide, high temperature polyimide, acrylate, UV-transparent fluoropolymer, fluorinated acrylate, silicone, polyethelene, polypropelene, acrylic polymethyl methacrylate, polystyrene, polyethelene terephthalate, polycarbonate polymers, CR-39, copolymers of styrene, Nylon, Teflon and their derivatives and combinations thereof.

31. A tube as claimed in claim 26 wherein the coating is transparent.

32. A tube as claimed in claim 26 in combination with a support plate, the tube having a proximal and a distal end and an inner diameter, being adapted to receive a first liquid, comprising at least one first region (“first sealing area”) and at least one second region (“second sealing area”) each being in contact with a means for fluid-tight closing the inner diameter of the tube within said first and second region and comprising in between said at least one first and said at least one second sealing areas at least one third region passing through a compartment of the support plate for receiving a second liquid.

33. The combination of claim 32 wherein the tube has at least one fourth region between said at least one third and the first or the second sealing area, said fourth region being amenable for visible or optical inspection (“inspection area”).

34. The combination of claim 33 wherein the tube follows a meandering track across the support plate such that multiple units of first and second sealing areas, third regions and inspection areas form a rectangular array.

35. The combination of claim 32 wherein more than one capillary tube is supported by the support plate, the tubes being arranged in such a manner that each follows the same track across the support plate.

36. The combination of claim 35 wherein the capillary tubes are arranged parallel.

37. The combination of claim 32 wherein the proximal and distal ends of the at least one capillary tube are located inside the support plate.

38. The combination of claim 32 wherein the proximal and distal ends of the at least one capillary tube are located outside the support plate.

39. The combination of claim 32 wherein the first and second sealing areas are provided as compartments containing a water immiscible sealing paste.

40. The combination of claim 32 wherein the said compartment of the support plate is in the form of a well through which the capillary tube passes.

41. The combination of claim 32 wherein the support plate is mounted on a motorised x/y/z adapter.

42. A method of using the combination claimed in claim 32 comprising the steps of:
(a) filling the at least one capillary tube with a solution of the biological molecule;
(b) filling individual precipitation reservoirs of the support plate for receiving a second liquid with a solution intended to provide crystallisation conditions for the biological molecule;
(c) interrupting and fluid-tightly closing the inner diameter of the at least one capillary tube within said first and second sealing areas, and
(d) forming a fluid connection between the precipitation reservoirs containing the second liquid and the inner volume of the capillary tube in which crystallisation of the biological molecule is to occur.

43. The method of claim 42 wherein the support plate comprises multiple units of first and second sealing areas and inspection areas arranged in a rectangular array and wherein steps (c) and/or (d) are carried out for units belonging to the same row or to the same column of the array simultaneously.

44. The method of claim 43 and including the further step (e) of visually or optically analysing the inspection areas for developing crystals of said biological molecule.

45. The method of claim 44 wherein step (e) is carried out by taking a first image from a location of said inspection area at a first point in time and a second image from the same location of said inspection area at a later point in time.

46. The method of claim 45 wherein the image information of said first image is subtracted from the image information of said second image.
